(19) United States
(12) Patent Application Publication
(10) Pub. No.: US 2006/0013896 A1
(43) Pub. Date: Jan. 19, 2006

(54) Methods of treating acute pain using diclofenac

(76) Inventors: Giorgio Reiner, Como (IT); Alberto Reiner, Como (IT)

Correspondence Address: KING & SPALDING LLP, 191 PEACHTREE STREET, N.E., 45TH FLOOR, ATLANTA, GA, 30303-1763, US

(21) Appl. No.: 11/180,996

(22) Filed: Jul. 13, 2005

Related U.S. Application Data
(63) Continuation-in-part of application No. 09/524,747, filed on Mar. 14, 2000.
Application No. 09/524,747 is a continuation-in-part of application No. 09/192,493, filed on Nov. 17, 1998, now abandoned.
Application No. 09/192,493 is a continuation of application No. PCT/EP97/02709, filed on May 15, 1997.

(30) Foreign Application Priority Data
May 17, 1996 (IT) MI96A000992

(51) Int. Cl. A61K 31/195

(52) U.S. Cl. 424/722; 514/567

(57) ABSTRACT

New pharmaceutical compositions for oral use containing diclofenac preferably together with alkali metal bicarbonates in amounts of from about 20 to about 80 by weight with respect to diclofenac are described. These compositions are entirely palatable and free from any unpleasant taste or other, side effects; in particular, these formulations permit to obtain in human patients higher Cₘₐₓ of the active principle and shorter Tₘₐₓ together with a lower coefficient of variation.

### RELATION TO PRIOR APPLICATIONS

The present application is a continuation-in-part of U.S. Ser. No. 09/524,747, filed Mar. 14, 2000 (pending), which is a continuation in part of U.S. Ser. No. 09/192,493, filed Nov. 17, 1998 (abandoned), which is a continuation of PCT/EP97/02709, filed May 15, 1997 with priority claimed to Italian App. No. MI96A000992, filed May 17, 1996. The contents of the foregoing applications are incorporated herein by reference as if fully set forth herein.

### FIELD OF INVENTION

The present invention relates to new immediate release pharmaceutical compositions containing [(2,6-dichloro-anilino)-2-phenyl]-2-acetic acid (more commonly known as diclofenac) in acid and/or salt form, and therapeutic regimens involving same for the treatment of acute pain.

### BACKGROUND OF INVENTION

Diclofenac is a non-steroidal drug which was invented at the end of the sixties by A. Sallmann and R. Pfister (NL-6,604,752 and U.S. Pat. No. 3,558,690 both to Ciba-Geigy) and whose structural formula is indicated below.

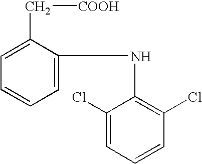

Diclofenac is widely dispensed and used owing to its well-known analgesic, anti-pyretic, anti-arthritic, anti-phlogistic and anti-rheumatic properties. It is generally taken orally in the form of normal tablets or tablets covered with coatings resistant to gastric juices, or rectally, or by injection, or topically.

The possibility of taking it in the form of sweets, tablets dissolving in the mouth, drages, chewing gum or other similar pharmaceutical forms or in formulations for the extemporary preparation of diclofenac-based aqueous solutions and/or suspensions would represent a different mode of administration which is definitely more suitable, especially for children and elderly persons.

Owing to its poor solubility in water, diclofenac is normally used in salt form; the salts of diclofenac customarily used are those of sodium, potassium or other alkali and alkaline earth metals, together with salts of organic nature, such as the salts of basic amino acids, such as lysine, arginine and omithine, or other pharmacologically acceptable organic bases which have the ability to render the resulting salt soluble in water.

The pharmaceutical compositions of the diclofenac salts for oral use are generally accompanied by side effects of not inconsiderable consequence: Diclofenac salts are in fact characterised by a particularly unpleasant and bitter taste and by the fact that they produce a sensation of strong astringency and cause an especially intense form of irritation in the buccal cavity, especially in the area of the larynx.

Although the first problem has been partly solved by using flavorings which are able in some manner to mask the taste, satisfactory solutions have still not been proposed for the two remaining problems.

Therefore, the pharmaceutical compositions containing diclofenac salts still have a poor palatability which limits their adoption and possible fields of application, despite the excellent therapeutic effect with which they are associated.

A second problem connected to diclofenac is that, when it is orally administered by means of immediate release formulations, the corresponding Tₘₐₓ (the time to the maximum plasma concentration) is usually located at about 1 hour since administration, this being of course a not completely satisfactory result when a prompt and strong analgesic/anti-pyretic effect is sought for. Furthermore, the corresponding coefficient of variation is normally in the range of 70-90%, which means that the Tₘₐₓ is strongly variable and dependent on the physical characteristics of the patient (Physicians' Desk Reference, 52 edition, 1998, page 1831). Attempts are therefore still being made in order to enhance the rate of absorption of diclofenac and to provide an earlier onset of the therapeutic effect (N. Davies, K. Anderson; Clinical Pharmacokinetics of Diclofenac, Clin. Pharmacokinet., 1997, September. 33(3)).

The object of the present invention is therefore that of providing a fully palatable formulation of diclofenac which is able to generate a more rapid, uniform and foreseeable release of the active principle if compared to the compositions known in the art and presently available on the market. For the purposes of the present invention Tₘₐₓ means the time to the maximum plasma concentration whereas Cₘₐₓ is the maximum plasma concentration of the active principle, namely diclofenac.

### DISCUSSION

It has now been found that, by adding alkali metal bicarbonates or mixtures thereof to the diclofenac in its acid and/or salt form, preferably in amounts of from 20 to 80% by weight based on the acid-form of diclofenac, pharmaceutical compositions can be obtained which are substantially free from the side effects mentioned above. The first object of the present invention is therefore represented by a pharmaceutical formulation for oral use containing diclofenac in acid and/or salt form together with alkali metal bicarbonates or mixtures thereof and customary excipients and adjuvants, wherein said alkali metal bicarbonates are preferably present in amounts of from 20 to 80% by weight based on the weight of diclofenac.

It has in fact been surprisingly demonstrated that the use of alkali metal bicarbonates in the above-mentioned ratio permits to achieve constant, reproducible and foreseeable blood levels of the active ingredient, with the consequent indisputable advantages from the therapeutic point of view; furthermore, it has also been found that the combined use of diclofenac together with alkali metal bicarbonates yields diclofenac-based pharmaceutical compositions in which the active ingredient is released more rapidly compared with normal formulations, bringing about higher blood levels and therefore a more immediate therapeutic effect; finally the so-obtained immediate release formulations are substantially palatable and free from aftertaste.

According to the preferred embodiment of the present invention, the amount of alkali metal bicarbonates to be added is comprised between 40 and 80% by weight, based on the weight of the acid-form diclofenac, whereas the alkali metal bicarbonates are selected from sodium and/or potassium bicarbonates, diclofenac being normally present in the form of its sodium and/or potassium salts. In various further embodiments, alkali metal carbonates and bicarbonates are employed in a weight ratio relative to the diclofenac of greater than about 1:5, 2:5, 2:1, 3:1 or 5:1. If desired, an upper limit on the buffer:diclofenac ratio can be placed at about 20:1, 10:1, 5:1, 1:1, 4:5 or 3:5. Ranges can be selected from any two of the foregoing values that are mathematically possible. In a preferred embodiment, the buffer:diclofenac weight ratio ranges from about 1:5 to about 4:5.

Although the compositions of the present invention are useful in chronic pain conditions such as rheumatoid arthritis, osteoarthritis, and ankylosing spondylitis; and periarticular disorders such as bursitis and tendonitis; they are particularly useful in the treatment of acute pain conditions, including soft tissue disorders such as sprains and strains, migraine attacks, and other painful conditions such as renal colic, acute gout, dysmenorrhoea, and following some surgical procedures.

It has also been found, and forms a second subject of the present invention, that the addition of flavoring substances selected from mint, aniseed, ammonium glycyrrhizinate and mixtures thereof to the compositions containing the diclofenac salts and alkali metal bicarbonates produces a synergistic effect which completely eliminates all the above-mentioned palatability/astringency effects, providing pharmaceutical compositions which are entirely palatable (and/or drinkable in the case of those used for the preparation of solutions and/or suspensions) and free from aftertaste.

The flavoring substances may be used as such or supported on inert materials, for example maltodextrin, in order to obtain a better distribution of the granulates and to facilitate excellent dispersibility of the flavoring in solution. Preferably, they are absorbed on maltodextrin with a power of 1 to 2000 and 1 to 1000.

The amount of flavoring substances in its pure form is also preferably from 1/5 to 3 times the weight of the acid-form diclofenac.

These flavoring substances are used in the implementation of the present invention without altering their organoleptic properties and without depriving them of their intrinsic qualities of flavorings which are liposoluble and generally oily in the pure state.

As it will be clear from the examples, the immediate release formulations for oral use of the present invention containing from 10 to 60 mg of diclofenac in acid and/or salt form together with alkali metal bicarbonates or mixtures thereof in amounts of from 20 to 80% by weight based on the weight of diclofenac permit to generate in human patients an average Cₘₐₓ of diclofenac comprised between 400 and 2500 ng/ml independently on the age, sex or weight of the patients themselves.

Secondly, the formulations according to the present invention permit to obtain in humans an average Tₘₐₓ of Diclofenac after 5-30 minutes since administration, generally 13-27, independently on the amount of diclofenac contained therein and also independently on the age, sex, weight of the patient.

Furthermore, the Tₘₐₓ of the formulations of the present invention show a coefficient of variation which is about 44-86% lower than the presently marketed formulations; this is evidently an extremely important result from the clinical point of view as it is now possible to have a therapeutic effect of diclofenac which is foreseeable, reproducible and independent of the sex, weight and health conditions of the patient.

Thus, the presently claimed diclofenac-based formulations permit to achieve a higher Cₘₐₓ in a shorter Tₘₐₓ and with a lower coefficient of variation if compared to the formulations available on the market, with therapeutic advantages which do not need to be commented.

Preferred Cmax and Tmax ranges for various formulations of the invention are set forth below in Table 1:

TABLE 1

| | Mean Cₘₐₓ (ng/ml) | Mean Tₘₐₓ (min) |
|---|---|---|
| 50 mg. | 1500-2100; 1750-2000; 1600-1900 | 5-35; 10-30; |
| diclofenac | | 12-25; 15-20 |
| tablet | | |
| 25 mg. | 700-1150; 750-950; 800-900; | 5-35; 10-30; |
| diclofenac | 850-1050; 900-1000 | 15-30; 15-25 |
| tablet | | |
| 12.5 mg. | 350-650; 400-600; 450-550 | 5-35; 10-30; 15-25 |
| diclofenac | | |
| tablet | | |
| 50 mg. | 1450-1850; 1500-2000; 1500-1750; | 5-35; 8-20; 10-18 |
| diclofenac | 1900-2500; 2000-2400; | |
| K powder | 2100-2300 | |
| sachet | | |

The amount of diclofenac used in the unit dosage forms of the present invention is preferably 12.5, 25, 50, 75 or 100 mg. According to the best mode for carrying out the present invention the pharmaceutical formulations will contain from 10 to 60 mg/dose of diclofenac in its potassium or sodium salt form together with 40 to 80% by weight of potassium or sodium bicarbonate based on the weight of diclofenac in its acid form, together with the usual excipients and adjuvants; even more preferably they will packaged as:
a sachet or tablet formulation containing 50 mg of diclofenac potassium salt and 22 mg of potassium bicarbonate, or a sachet or tablet containing 100 mg., 75 mg., 25 mg. or 12.5 mg of diclofenac potassium and potassium bicarbonate in an amount of 44 wt. % based on the weight of the diclofenac.
a sachet or tablet formulation containing 50 mg of diclofenac sodium salt and 19 mg of sodium bicarbonate, or a sachet or tablet containing 100 mg., 75 mg., 25 mg. or 12.5 mg of diclofenac sodium and sodium bicarbonate in an amount of 38 wt. % based on the weight of the diclofenac.

It will be by the way evident to any skilled in this art that the present formulations can also be used as immediate release layers of multilayered release pharmaceutical formulations containing diclofenac as one of the active ingredients; said formulations are therefore a further object of the present invention.

### EXAMPLES

The following Examples are given purely by way of non-limiting illustration.

### Example 1

### Composition Dissolving Instantly in Water

| Active ingredients | | | |
|---|---|---|---|
| 1) | Diclofenac potassium salt*: | 50 | mg |
| 2) | Potassium bicarbonate: | 22 | mg |
| 3) | Mint flavoring on maltodextrin (1:2000)**: | 60 | mg |
| 4) | Aniseed flavoring on maltodextrin (1:1000)***: | 104 | mg |
| | Excipients and adjuvants | | |
| 5) | Saccharin: | 4 | mg |
| 6) | Aspartame: | 10 | mg |
| 7) | Mannitol: | 50 | mg |
| 8} | Saccharose****q.s.: | 2 | g |

*If it is desired to prepare compositions based on diclofenac sodium salt, it is advantageous to use sodium bicarbonate in a quantity of approximately 38% by weight based on the weight of the diclofenac sodium salt present.
Sodium carbonate may also be added to the sodium bicarbonate, maintaining the following optimum proportions: 27% of sodium bicarbonate and 4-5% of sodium carbonate, always based on the amount by weight of diclofenac sodium salt present.
**The title of the pure mint essence, as obtained according to the Dean-Stark method, is of 18% by weight; the related amount is therefore in this case of 10.8 mg.
***The title of the pure anise essence, as obtained according to the Dean-Stark method, is of 14.5% by weight, the related amount is therefore in this case of 16 mg.
****The presence of saccharose is not strictly necessary; in its absence, a composition having a very limited granulate content is obtained which is perfectly 20 soluble in contact with water. In that case, nothing is changed from the point of view of tolerability in contact with the mucosa and. from the point of view of the palatability of the drinkable solution.

Preparation

Components 1, 2, 5, 6 and 7 are mixed in a suitable mixer, and the mixture so obtained is wetted with 95% ethanol. Granulation is carried out with a 66 mm mesh and the granulate is preferably dried in current of air.

Components 3, 4 and 8, which have already been granulated using a mesh of the same granulometry, are then added and the whole is mixed.

The mixture is then introduced into a metering machine for filling packets or similar containers.

### Example 2

### Tablet for Dissolving in the Mouth

| Active ingredients | | |
|---|---|---|
| 1) | Diclofenac potassium salt*: | 50 mg |
| 2) | Potassium bicarbonate: | 35 mg |
| 3) | Mint flavoring on maltodextrin** | 50 mg |
| | (1:2000) + gum arabic (E 414): | |
| 4) | Aniseed flavoring (1:1000) | 120 mg |
| | on maltodextrin*** + silicon | |
| | dioxide(E551): | |
| | Excipients and adjuvants | |
| 5) | Saccharin: | 50 mg |
| 6) | Aspartame: | 12 mg |
| 7) | Mannitol: | 20 mg |
| 8) | Saccharose****: | 300 mg |

*to **** see Example 1

### Example 3

### Gum Tablet

| Active ingredients | | |
|---|---|---|
| 1) | Diclofenac potassium salt*: | 50 mg |
| 2) | Potassium bicarbonate: | 35 mg |
| 3) | Mint flavoring on maltodextrin**: | 30 mg |
| 4) | Aniseed flavoring on maltodextrin***: | 80 mg |
| | Excipients and adjuvants | |
| 5) | Mannitol: | 30 mg |
| 6) | Menthol: | 0.01 mg |
| 7) | Gum base: | 600 mg |
| 8) | Sorbitol: | 700 mg |
| 9) | Saccharin: | 3 mg |
| 10) | Hydroxypropylmethylcellulose: | 33 mg |
| 11) | Coloring agent: | 7 mg |

*to *** see Example 1

### Example 4

### Comparative Test

The packaged composition containing 50 mg of diclofenac potassium of Example 1 (formulation C) was subjected to a pharmacokinetic test for comparison with a similar composition not containing alkali metal carbonates and bicarbonates (formulation B), and with a second composition in tablet form (formulation A) produced by Ciba-Geigy (Voltaren Rapid®), also in this case not containing alkali metal carbonates and bicarbonates, both formulations A and B containing 50 mg of diclofenac potassium.

This comparative evaluation was carried out on the same 6 healthy volunteers in accordance with the experimental plan described hereinafter.
Experimental scheme: Single-dose study using three methods in randomized 15 cross-over with a wash-out of three days.
Sampling times: 0 h (before administration), 5 min, 10 min, 30 min, 45 min, 1 h, 1.5 h, 2 h, 3 h, 4 h, 6 h, 8 h, after each administration.
Blood sample treatment: 8MI in heparinised test tubes, centrifugation for 15 min at 1500 rev/min, subdivided into two fractions and subsequently frozen at −200 C.
Times: wash-out of two days between treatments.
Determination method: HPLC, with internal standard, sensitivity 10 ng/ml

Analysis Method
Column: Nova Pak C18, 3.9×150 mm, 4 um Waters S.p.A.—Vimodrone, Italy.
Eluant: NaH2PO4 0.01 M+0.1% TEA, pH 3.0 (H3PO4)/acetonitrile, 60/40.
Flow: 1.2 ml/min
Detection: UV/275 nm
Temperature: 30° C.
Injection: 50 al
Analysis time: 16 min.

Sample Preparation

10 al of the internal standard methanolic solution, and flufenamic acid (corresponding to 1320 ng) are added to 1 ml of defrosted plasma in 10 ml glass test tubes. The tubes are agitated in a Vortex mixer for 1 minute. 0.5 ml of a 0.5N HCl/1N NaCl solution is added. The whole is agitated in a Vortex mixer for 1 minute. 6 ml of a 95/5 n-hexane/isopropanol solution are added.

The mixture is then agitated in the Vortex mixer for a further 15 minutes. Centrifugation is carried out at 3000 rev/min for 15 minutes and the organic phase is transferred to fresh 10 ml glass test tubes and evaporated to dryness in a centrifugal evaporator under vacuum at ambient temperature. The whole is taken up in 200 al of a 70/30 acetonitrile/water solution, and the precipitate is dissolved under ultrasound for 2 minutes.

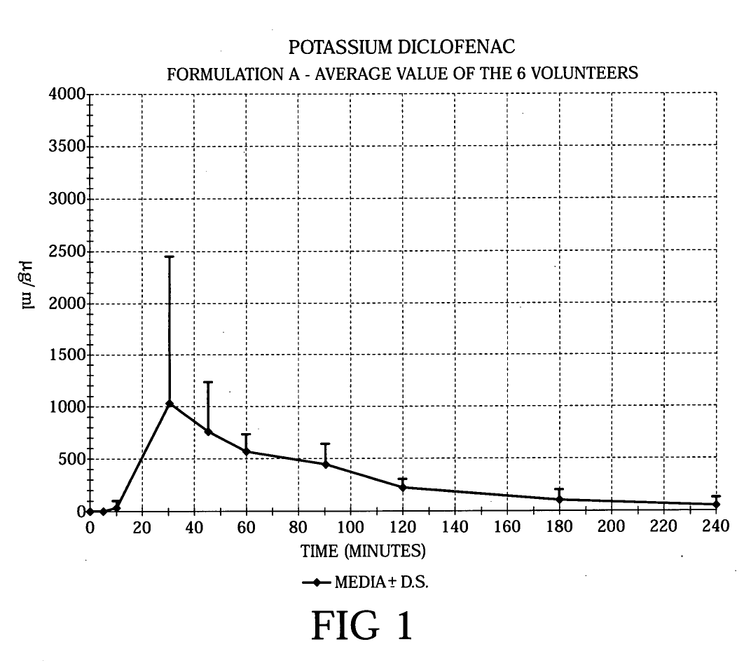

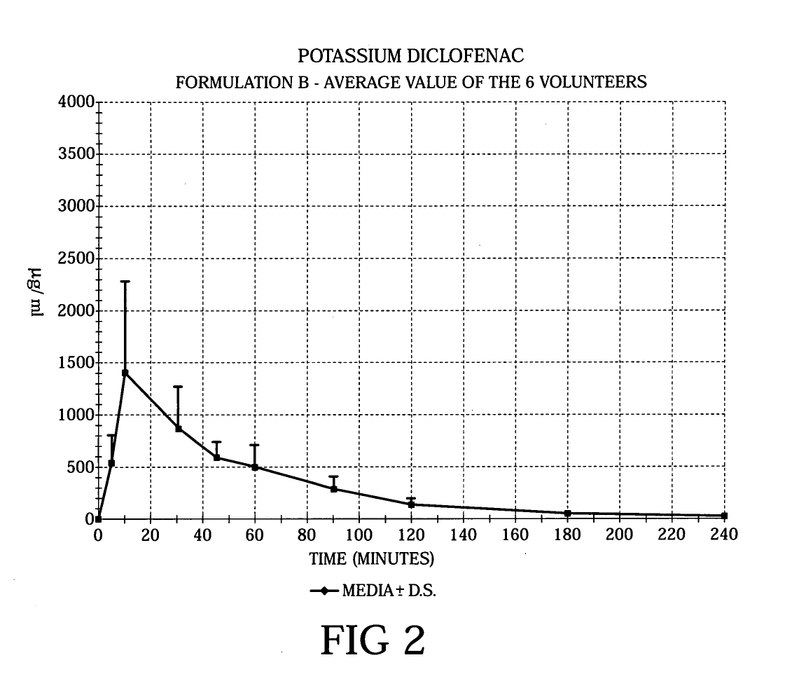

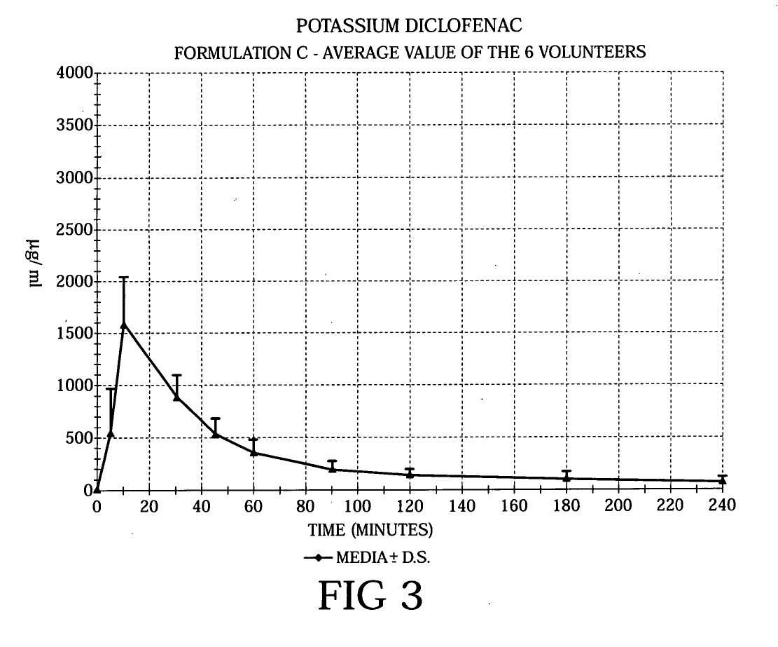

FIGS. 1, 2 and 3 show the concentrations of diclofenac in the blood of the six volunteers as regards formulations A, B (Ciba-Geigy comparative formulations) and C (formulation corresponding to the composition of Example 1), respectively.

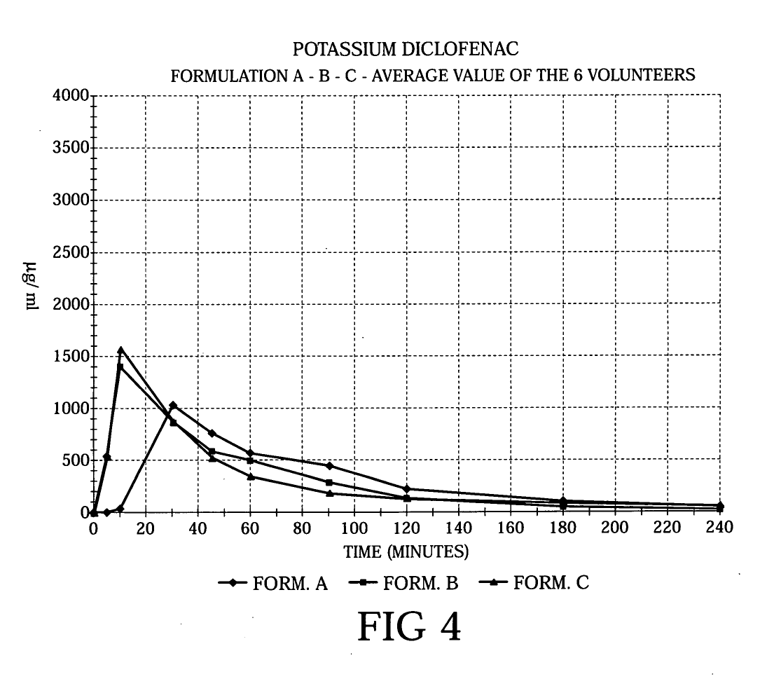

As will be appreciated, the blood concentration of the formulation of the present invention has, compared with the comparative formulations, a more constant and uniform pattern. This characteristic is also found in FIGS. 4, 5 and 6 which show the average values corresponding to the blood levels of the six volunteers together with the corresponding standard deviation.

The result is clear and surprising: compared with the sample compositions, the compositions of the present invention permit constant, reproducible and foreseeable blood levels of the active ingredient, irrespective of the characteristics of the volunteer (weight, age, etc), with the consequent indisputable advantages from the therapeutic point of view.

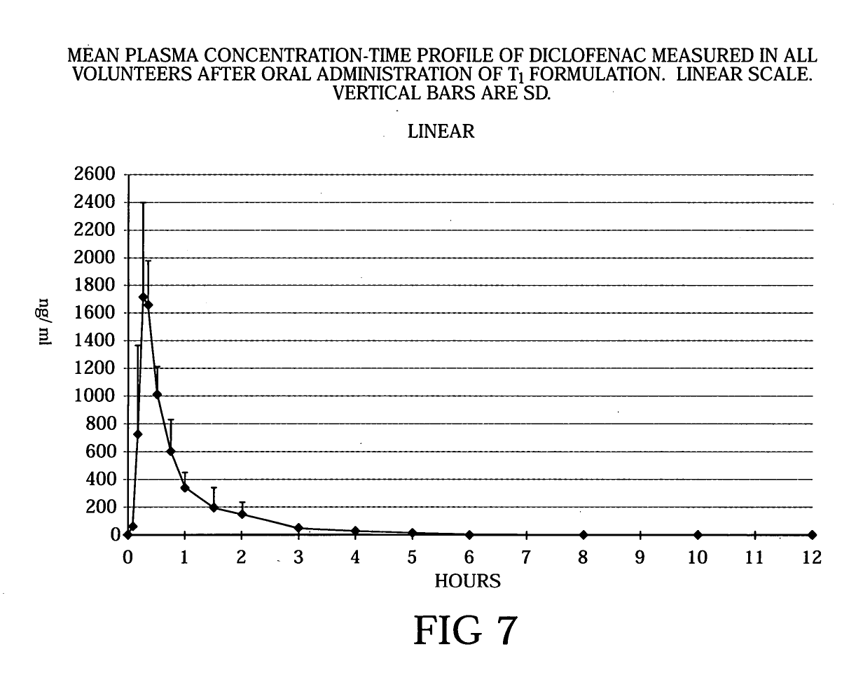

Finally, FIG. 7 shows, by comparison, the graphs relating to the average values of the six volunteers (that is to say, the preceding FIGS. 4, 5 and 6); as will be noted, the formulation of the present invention permits, in addition to the advantages already mentioned, the attainment of a blood peak higher than that of the other formulations.

### Example 5

### Two Layered Tablet (Fast and Slow Release)

| | |
|---|---|
| Fast release layer | |
| 1) Diclofenac potassium salt: | 15 mg |
| 2) Potassium bicarbonate: | 30 mg |
| 3) Lactose: | 13.2 mg |
| 4) Maize starch (intragranular): | 6 mg |
| 5) Methyl cellulose: | 0.12 mg |
| 6) Sodium laurylsulfate: | 0.06 mg |
| 7) Maize starch (extragranular): | 9 mg |
| 8) Crospovidone: | 0.6 mg |
| 9) Sodium carboxymethylstarch: | 1.5 mg |
| 10) Magnesium stearate: | 2.7 mg |
| 11) Colloidal silicon dioxide: | 0.6 mg |
| Slow release layer | |
| 1) Diclofenac potassium salt: | 70 mg |
| 2) Potassium bicarbonate: | 30.8 mg |
| 3) Lactose: | 32.2 mg |
| 4) Polyvinylpyrrolidone: | 1.16 mg |
| 5) Hydroxypropylmethylcellulose: | 70 mg |
| 6) Magnesium stearate: | 0.84 mg |
| 7) Colloidal silicon dioxide: | 0.21 mg |
| 8) Talc: | 3.92 mg |
| 9) Polyethylene glycol: | 0.56 mg |

### Example 6

### Drops

| | |
|---|---|
| 1) Diclofenac potassium salt: | 75 g |
| 2) Methyl p-oxybenzoate: | 2.7 g |
| 3) Propyl p-oxybenzoate: | 0.3 g |
| 4) Aspartame: | 37.5 g |
| 5) Potassium bicarbonate: | 37.5 g |
| 6) Glycerol: | 300 g |
| 7) Ethyl alcohol: | 450 g |
| 8) Water q.s.: | 1500 g |
| Possible modifications: | |
| a) Addition of sodium metabisulfite (0.06%) | |
| b) Addition of sodium metabisulfite (0.06%) | |
| Mint flavoring (1.25%) | |
| Strawberry flavoring (0.75%) | |

### Example 7

### Drops

| | |
|---|---|
| 1) Diclofenac potassium salt: | 37.5 g |
| 2) Methyl p-oxybenzoate: | 2.7 g |
| 3) Propyl p-oxybenzoate: | 0.3 g |
| 4) Aspartame: | 37.5 g |
| 5) Potassium bicarbonate: | 18.75 g |
| 6) Saccharin: | 6.0 g |
| 7) Glycerol: | 300 g |
| 8) Ethyl alcohol: | 450 g |
| 9) Water q.s.: | 1500 g |
| Possible modifications: | |
| a) Addition of sodium metabisulfite (0.03%) | |
| b) Addition of sodium metabisulfite (0.03%) | |
| Mint flavoring (1.25%) | |
| Strawberry flavoring (0.75%) | |

### Example 8

### Mouthwash

| | | |
|---|---|---|
| 1) Diclofenac potassium salt: | 0.75 | g |
| 2) Glycerol: | 50 | g |
| 3) Sorbitol: | 12 | g |
| 4) Saccharin: | 0.5 | g |
| 5) Aspartame: | 1.0 | g |
| 6) Methyl p-oxybenzoate: | 0.5 | g |
| 7) Propyl p-oxybenzoate: | 0.1 | g |
| 8) Mint flavoring: | 1.0 | g |
| 9) Ethyl alcohol: | 100 | g |
| 10) Potassium bicarbonate: | 0.33 | g |
| 11) Water q.s.: | 500 | ml |

### Example 9

### Gum-Paste

| | | |
|---|---|---|
| 1) Diclofenac potassium salt: | 5.0 | g |
| 2) Glycerol: | 630 | g |
| 3) Sodium benzoate: | 5.0 | g |
| 4) Silica (Wessalon S ® - Degussa): | 120 | g |
| 5) Silica (Siddent 9 ® - Degussa): | 80 | g |
| 6) Cellulose gum: | 3.0 | g |
| 7) Polyethyleneglycol 600: | 30 | g |
| 8) Sodium lauroyl sarcosinate (or sodium lauryl sulfate): | 60 | g |
| 9) Mint flavoring: | 10 | g |
| 10) Sodium saccharin: | 1.0 | g |
| 11) Aspartame: | 3.0 | g |
| 12) Potassium bicarbonate: | 2.2 | g |
| 13) Water q.s.: | 1 | kg |

### Example 10

### Tooth-Paste

| | | |
|---|---|---|
| 1) Diclofenac potassium salt: | 5.0 | g |
| 2) Glycerol: | 630 | g |
| 3) Sodium benzoate: | 5.0 | g |
| 4) Silica (Wessalon S ® - Degussa): | 20 | g |
| 5) Silica (Siddent 9 ® - Degussa): | 80 | g |
| 6) Cellulose gum: | 3.0 | g |
| 7) Polyethylenglycol 600: | 30 | g |
| 8) Sodium lauroyl sarcosinate (or sodium lauryl sulfate): | 60 | g |
| 9) Mint flavoring: | 10 | g |
| 10) Sodium saccharin: | 1.0 | g |
| 11) Aspartame: | 3.0 | g |
| 12) NaF: | 1.0 | g |
| 13) Na₂FPO₃ | 4.0 | g |
| 14) Potassium bicarbonate: | 2.2 | g |
| 15) Water q.s.: | 1 | kg |

### Example 11

### Tablet

| | | |
|---|---|---|
| 1) Diclofenac potassium salt: | 50 | mg |
| 2) Mannitol: | 50 | mg |
| 3) Potassium bicarbonate: | 22 | mg |
| 4) Maize starch (intragranular): | 10 | mg |
| 5) Methyl cellulose: | 0.2 | mg |
| 6) Sodium laurylsulfate: | 0.1 | mg |
| 7) Maize starch (extragranular): | 15 | mg |
| 8) Crospovidone: | 1.0 | mg |
| 9) Sodium carboxymethylstarch: | 2.5 | mg |
| 10) Magnesium stearate: | 4.5 | mg |
| 11) Colloidal silicon dioxide: | 10 | mg |

### Example 12

### Comparative Test

In the present experiment a sachet formulation containing 50 mg of diclofenac potassium was compared to a bioequivalent sugar coated fast release tablet also containing 50 mg of diclofenac potassium, produced and marketed in Italy by Novartis as Cataflam®.

The sachet formulation according to the present invention had the following composition:

| | | |
|---|---|---|
| 1) diclofenac potassium salt: | 50 | mg |
| 2) Potassium bicarbonate: | 22 | mg |
| 3) Mint flavor: | 50 | mg |
| 4) Anice flavor: | 100 | mg |
| 5) Saccharin sodium: | 4 | mg |
| 6) Aspartame: | 10 | mg |
| 7) Mannitol: | 50 | mg |
| 8) Sucrose sugar crystals: | 1714 | g |

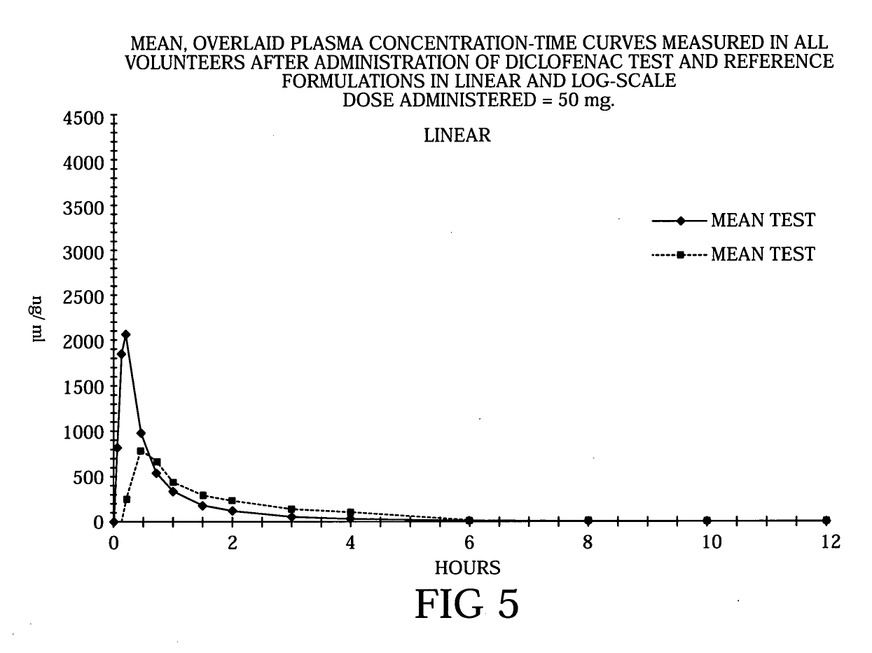

The above test formulation and the Cataflam® formulation were administered as a single dose to 24 healthy volunteers of both sexes. The pharmacokinetic parameters obtained with the two different formulations are reported in table 1 and in FIG. 5. As it will be easily appreciated, the rate of absorption was considerably faster with the sachet formulation of the present invention than with Cataflam®, the sachet formulation having a higher average Cₘₐₓ (2213 vs 1071 ng/ml) and a shorter average Tₘₐₓ (0.228 vs 0.885 hours); furthermore, the Tₘₐₓ of the sachet formulation shows a coefficient of variation lower than the reference formulation (16% vs 97%), this being an extremely important result from the clinical point of view regarding the healing of the pain in terms of quick time and repeatability inter-subjects in order to reach the Cₘₐₓ.

### Example 13

### Comparative Test

Following to the excellent results obtained in example 12, two tablet formulations containing 12.5 or 25 mg. of diclofenac sodium salt and potassium bicarbonate (in the same weight ratio) have been prepared.

The tablet formulations had the following composition (in mg):

| Cores | | |
|---|---|---|
| Diclofenac sodium | 12.5 | 25 |
| Mannitol | 25 | 50 |
| Lactose monohydrate | 23.75 | 47.5 |
| Potassium bicarbonate | 5.5 | 11 |
| Maize starch | 22.5 | 45 |
| Methylcellulose | 0.075 | 0.15 |
| Sodium laurylsulphate | 0.125 | 0.25 |
| Crospovidone | 3 | 6 |
| Ultramyl | 5 | 10 |
| Colloidal silica | 0.55 | 1.1 |
| Cellulose microcrystalline | 0.5 | 1 |
| Magnesium stearate | 1.5 | 3 |
| Purified water q.s. | 100 | 200 |
| Coating | | |
| Opadry OY-3 5009 red | 2 | 4 |
| Macrogol 400 | 0.25 | 0.5 |

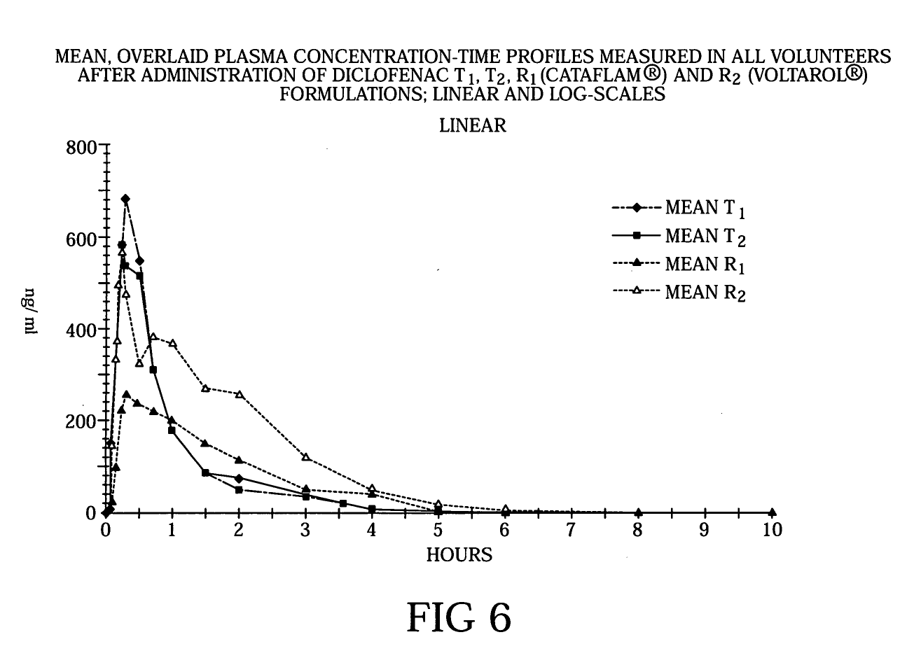

A four-way comparative bioavailability study was carried out on 18 healthy volinteers of both sexes in order to evaluate the in vivo results of the pharmacokinetic profiles of the present formulations if compared to those of bioequivalent fast release formulations such as Cataflam® (25 mg of diclofenac potassium) and Voltarol® (50 mg of diclofenac sodium), both by Novartis. The results, which are summarized in FIG. 6, indicate that Tₘₐₓ is prompter with the present formulations (T1=26 min, T2=24.6 min vs R1=71.4 min and R2=40.8 min) and that Cₘₐₓ is higher (T1=847 ng/ml and T2=861 ng/ml vs R1=452 ng/ml and R2=703 ng/ml); furthermore, the Tₘₐₓ of both present formulations shows a coefficient of variation lower than reference formulations (T1=46% and T2=49% vs R1=87% and R2=96%).

### Example 14

### Comparative Test

A further comparative test was carried out on immediate release formulations according to the present invention, containing 50 mg of diclofenac potassium and 22 mg of potassium bicarbonate, manufactured with different that is, respectively: T1=wet granulation using alcohol, T2=dry granulation by direct compression. The composition in mg of the two formulations is herebelow reported:

| | | |
|---|---|---|
| Diclofenac potassium | 50 | 50 |
| Potassium bicarbonate | 22 | 22 |
| Mannitol/pearlitol 400 DC | 119.9 | |
| Mannitol EP cf | | 50 |
| Maize starch | | 25 |
| Methocel A4C | | 0.2 |
| Sodium laurylsulphate | 0.1 | 0.1 |
| Polyplasdone XL | 6 | 1 |
| Ultramyl | | 2.5 |
| Magnesium stearate | 2 | 4.5 |
| Silicium aerosil | | 1 |
| Core mass | 200 | 156.3 |

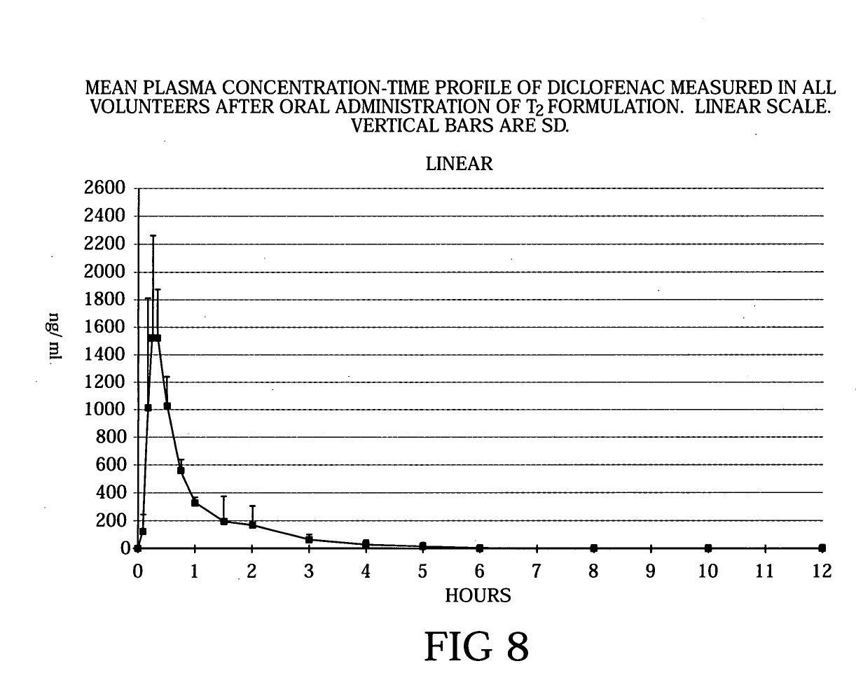

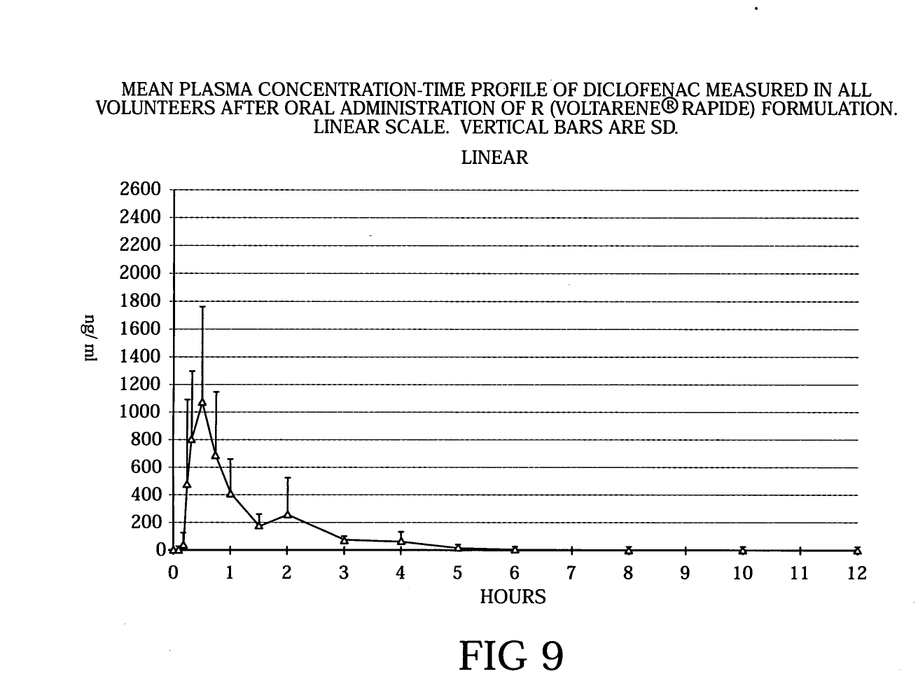

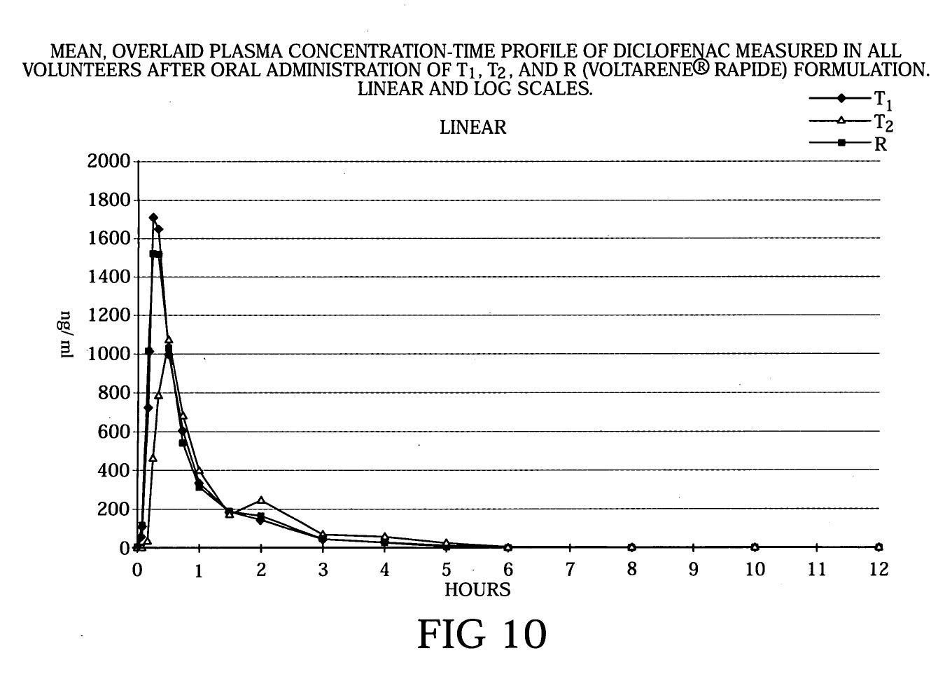

A comparative bioavailabilty study was carried out on 6 healthy volunteers of both sexes in order to evaluate the in vivo results of the pharmacokinetic profiles of the present formulations if compared to those of a bioequivalent fast release formulation such as Voltaren Rapid® (50 mg of diclofenac potassium), both by Novartis. The results, which are reported in FIGS. 7-10 are also in this case excellent: the Tₘₐₓ is in fact prompter with the present formulations (T1=18.6 min, T2=16.8 min vs R1 40.8 min) and the Cₘₐₓ is higher (T1=1878.3 ng/ml and T2=1744.8 ng/ml vs R1 1307 ng/ml); furthermore, also in this case the Tₘₐₓ of both present formulations shows a coefficient of variation lower than reference formulation (T1=12.9% and T2=25% vs R1=95.6%).

### Example 15

### 50 mg. Diclofenac K Tablet Comparison

Test Formulations:
   T1: Diclofenac potassium 50 mg film-coated tablets, alcohol granulation
   T2: Diclofenac potassium 50 mg film-coated tablets, dry granulation
Reference Formulation: Diclofenac potassium, 50 mg film-coated tablets, Voltarene® Rapid by Novartis Pharma
Study design: Single dose, 3-way, crossover randomised on 6 healthy volunteers
Blood samples drawn: 0 (pre-dose), 5, 10, 15, 20, 30, 45, 60, 90 min, 2, 3, 4, 5, 6, 8, 10, 12 h

Assay: LC-MS-MS //LOQ 5 ng/ml

| | | | Reference, K salt, 50 mg, |
|---|---|---|---|
| | T1, K salt, 50 mg, tablets | T2, K salt, 50 mg, tablets | Voltaren ® Rapid tablets |
| Description | Diclofenac potassium | Diclofenac potassium | Diclofenac potassium |
| | 50 mg film-coated tablets | 50 mg film-coated tablets | 50 mg film-coated tablets |
| | (by alcoholic granulation) | (by direct compression) | |
| Active | Diclofenac potassium mg 50 | Diclofenac potassium mg 50 | Diclofenac potassium mg 50 |
| ingredient | | | |
| Excipients | Potassium bicarbonate mg 22 | Potassium bicarbonate mg 22 | Calcium phosphate |
| | Mannitol mg 50 | Mannitol 400 mg 119.9 | Saccharose |
| | Maize starch mg 25 | Sodium laurylsulfate mg 0.1 | Maize starch |
| | Hydroxypropylmethylcellulose mg 0.2 | Polyvinylpyrrolidone mg 6 | Talc |
| | Sodium laurylsulfate mg 0.1 | Magnesium stearate mg 2 | Sodium carboxymethylcellulose |
| | Polyvinylpyrrolidone mg 1 | Film Coating Opadry Clear | Colloidal anhydrous silicium |
| | Sodium starch glycollate mg 2.5 | (HPMC 2910, | Polyvinylpyrrolidone |
| | Magnesium stearate mg 4.5 | polyethyleneglycol 400) mg 4 | Microcrystalline cellulose |
| | Silicium aerosil FK 160 mg 1 | | Magnesium stearate |
| | Coating Opadry Clear (HPMC 2910 | | Polyethylenglycole |
| | and polyethyleneglycol 400) mg 4 | | Titanidioxide (E171) |
| | | | Iron oxide red (E172) |
| Total weight | 160.3 mg | 204 mg | |

| | | PK results | | |
|---|---|---|---|---|
| | | Test 1 (K, tablets 50 mg) | Test 2 (K, tablets 50 mg) | Reference (K, tablets 50 mg) |
| Cₘₐₓ | Mean | 1873.30 | 1744.8 | 1307.0 |
| | SD | 553.80 | 572.3 | 558.4 |
| | CV % | 29.5 | 32.8 | 42.7 |
| | Min | 1228.9 | 1057.4 | 581.8 |
| | Max | 2516.5 | 2468.9 | 1935.5 |
| AUC | Mean | 1219 | 1237 | 1168 |
| | SD | 246 | 276 | 282 |
| | CV % | 20.2 | 22.3 | 24.1 |
| | Min | 874 | 848 | 913 |
| | Max | 1615 | 1668 | 1642 |
| tₘₐₓ | Mean | 0.31 h (18.6 min) | 0.28 h (16.8 min) | 0.68 h (40.8 min) |
| | SD | 0.04 | 0.07 | 0.65 |
| | CV % | 12.9 | 25.0 | 95.6 |
| | Min | 0.25 h (15 min) | 0.17 h (10.2 min) | 0.25 h (15 min) |
| | Max | 0.33 h (19.8 min) | 0.33 h (19.8 min) | 2.00 h (120 min) |

### Example 16

### 50 and 25 mg. Diclofenac K Tablet Comparison

Test Formulations:
   T1: Diclofenac potassium 25 mg film-coated tablets
   T2: Diclofenac potassium 50 mg film-coated tablets
Reference Formulation: Diclofenac potassium, 50 mg film-coated tablets, Voltarene® Rapid by Novartis Pharma
Study design: Single dose, 3-way, crossover randomised on 24 healthy volunteers
Blood samples drawn: 0 (pre-dose), 5, 10, 15, 20, 30, 45, 60, 90 min, 2, 3, 4, 5, 6, 8, 10, 12 h

Assay: LC-MS-MS //LOQ 5 ng/ml

| | | | Reference, K salt, 50 mg, |
|---|---|---|---|
| | T1, K salt, 25 mg, tablets | T2, K salt, 50 mg, tablets | Voltaren ® Rapid tablets |
| Description | Diclofenac potassium 25 mg film- | Diclofenac potassium 50 mg | Diclofenac potassium 50 mg film- |
| | coated tablets | film-coated tablets | coated tablets |
| Active | Diclofenac potassium mg 25 | Diclofenac potassium mg 50 | Diclofenac potassium mg 50 |
| ingredient | | | |
| Excipients | Potassium bicarbonate mg 11 | Potassium bicarbonate mg 22 | Calcium phosphate |
| | Mannitol 400, mg 58.2 | Mannitol 400, mg 116.4 | Saccharose |
| | Sodium laurylsulfate mg 0.05 | Sodium laurylsulfate mg 0.1 | Maize starch |
| | Polyvinylpyrrolidone mg 3 | Polyvinylpyrrolidone mg 6 | Talc |
| | Polyethylenglycole mg 0.75 | Polyethylenglycole mg 1.5 | Sodium carboxymethylcellulose |
| | Magnesium stearate mg 2 | Magnesium stearate mg 4 | Colloidal anhydrous silicium |
| | Film Coating Opadry Clear (HPMC | Film Coating Opadry Clear | Polyvinylpyrrolidone |
| | 2910, polyethyleneglycol 400) mg 2 | (HPMC 2910, | Microcrystalline cellulose |
| | | polyethyleneglycol 400) mg 4 | Magnesium stearate |
| | | | Polyethylenglycole |
| | | | Titanidioxide (E171) |
| | | | Iron oxide red (E172) |
| Total weight | 102 mg | 204 mg | |

| | | PK results | | |
|---|---|---|---|---|
| | | | T2 (K, film-coated tablets | |
| | | T1 (K, tablets 25 mg) | 50 mg) | Reference (K, tablets 50 mg) |
| Cₘₐₓ | Mean | 940.2 (1880.4)* | 1766.7 | 1339.6 |
| | SD | 387.0 | 1020.2 | 627.5 |
| | CV % | 41.2 | 57.7 | 46.8 |
| | Min | 228.5 | 317.3 | 336.5 |
| | Max | 1595.4 | 4516.9 | 2655.4 |
| AUC | Mean | 611.81 (1223.63)* | 1267.67 | 1286.43 |
| | SD | 144.76 | 356.46 | 351.22 |
| | CV % | 23.7 | 28.1 | 27.3 |
| | Min | 380.13 | 681.89 | 852.09 |
| | Max | 919.81 | 2123.22 | 2185.01 |
| tₘₐₓ | Mean | 0.354 h (21.2 min) | 0.489 h (29.8 min) | 0.847 h (50.8 min) |
| | SD | 0.119 | 0.366 | 0.887 |
| | CV % | 33.6 | 78.8 | 104.7 |
| | Min | 0.250 h (15 min) | 0.167 h (10 min) | 0.333 h (20 min) |
| | Max | 0.750 h (45 min) | 1.5 h (90 min) | 4 h (240 min) |

*values normalized to the dose of 50 mg

### Example 17

### 25 mg. Diclofenac Na Tablet and Sachet Comparison

Test Formulations:
   T1: Diclofenac sodium 25 mg sachets
   T2: Diclofenac sodium 25 mg film-coated tablets
Reference Formulation: Diclofenac sodium, 25 mg film-coated tablets, Novapirina® by Novartis Consumer Health
Study design: Single dose, 3-way, crossover randomised on 24 healthy volunteers
Blood samples drawn: 0 (pre-dose), 10, 20, 30, 45, 60, 75, 90, 105 min, 2, 2.5, 3, 4, 6, 8, 10 h

Assay: LC-MS-MS //LOQ 0.5 ng/ml

| | | | Reference, Na salt, |
|---|---|---|---|
| | Na salt, 25 mg, sachets | Na salt, 25 mg, tablets | Novapirina ® tablets |
| Description | NA | Diclofenac sodium 25 mg | Diclofenac sodium 25 mg film- |
| | | film-coated tablets | coated tablets |
| Active | NA | Diclofenac sodium 25 mg | |
| ingredient | | | |
| Excipients | NA | Potassium bicarbonate mg 11 | Colloidal silicium |
| | | Mannitol SD200, mg 58.25 | Cellulose |
| | | Sodium laurylsulfate mg 0.25 | Lactose |
| | | Polyvinylpyrrolidone mg 3 | Magnesium stearate |
| | | Magnesium stearate mg 1 | Polyvidone |
| | | Glyceryl Dibehenate mg 1.5 | Sodium |
| | | Film Coating Opadry Clear | carboxymethylcellulose |
| | | (HPMC 2910, | Hydroxypropylmethylcellulose |
| | | polyethyleneglycol 400) mg 2 | Polysorbate |
| | | | Talc |
| | | | Titanidioxide (E171) |
| Total weight | NA | 102 mg | |

| | | PK results | | |
|---|---|---|---|---|
| | | Test 1 (Na, sachets 25 mg) | Test 2 (Na, tablets 25 mg) | Reference (Na, tablets 25 mg) |
| Cₘₐₓ | Mean | NA | 863 | 554 |
| | SD | NA | 373 | 255 |
| | CV % | NA | 43.2 | 87 |
| | Min | NA | 382 | 222 |
| | Max | NA | 1620 | 1360 |
| AUC | Mean | NA | 632 | 581 |
| | SD | NA | 175 | 151 |
| | CV % | NA | 27.7 | 26 |
| | Min | NA | 375 | 355 |
| | Max | NA | 974 | 860 |
| tₘₐₓ | Mean | NA | 0.32 h (19.2 min) | 0.67 h (40.2 min) |
| | SD | NA | 0.07 | 0.62 |
| | CV % | NA | 21.9 | 92.5 |
| | Min | NA | 0.17 h (10.2 min) | 0.17 h (10.2 min) |
| | Max | NA | 0.50 h (30 min) | 2.50 h (150 min) |

### Example 18

### 50 mg. Diclofenac K Tablet Comparison

Test Formulation: Diclofenac potassium 50 mg film-coated tablets
Reference Formulation: Diclofenac potassium, 50 mg film-coated tablets, Voltfast® by Novartis Pharma
Study design: Single dose, 2-way, crossover randomised on 26 healthy volunteers
Blood samples drawn: 0 (pre-dose), 10, 20, 30, 45, 60, 90 min, 2, 3, 4, 5, 6, 8, 10, 12 h

Assay: LC-MS-MS //ILOQ 3.3 ng/ml

| | | Reference, K salt, 50 mg, |
|---|---|---|
| | K salt, 50 mg, tablets | Voltfast ® tablets |
| Description | Diclofenac potassium | Diclofenac potassium 50 mg |
| | 50 mg film-coated tablets | film-coated tablets |
| Active | Diclofenac potassium | Diclofenac potassium mg 50 |
| ingredient | mg 50 | |
| Excipients | Potassium bicarbonate | Calcium phosphate |
| | mg 22 | Saccharose |
| | Mannitol 400, mg 116.4 | Maize starch |
| | Sodium laurylsulfate | Talc |
| | mg 0.1 | Sodium carboxymethylcellulose |
| | Polyvinylpyrrolidone | Colloidal anhydrous silicium |
| | mg 6 | Polyvinylpyrrolidone |
| | Polyethylenglycole | Microcrystalline cellulose |
| | mg 1.5 | Magnesium stearate |
| | Magnesium stearate mg 4 | Polyethylenglycole |
| | Film Coating Opadry | Titanidioxide (E171) |
| | Clear (HPMC 2910, | Iron oxide red (E172) |
| | polyethyleneglycol 400) | |
| | mg 4 | |
| Total weight | 204 mg | |

| | | PK result | |
|---|---|---|---|
| | | Test (K, tablets 50 mg) | Reference (K, tablets 50 mg) |
| Cₘₐₓ | Mean | 1768.6 | 1386.5 |
| | SD | 771.6 | 693.3 |
| | CV % | 43.6 | 50.0 |
| | Min | 514.3 | 300.2 |
| | Max | 3726.4 | 2744 |
| AUC | Mean | 1248 | 1220.2 |
| | SD | 326 | 352.7 |
| | CV % | 26.1 | 28.9 |
| | Min | 661.8 | 609 |
| | Max | 1918.4 | 1971.3 |
| tₘₐₓ | Mean | 0.455 h (27.3 min) | 0.904 h (54.24 min) |
| | SD | 0.275 | 0.838 |
| | CV % | 60.0 | 92.7 |
| | Min | 0.166 h (10 min) | 0.333 h (20 min) |
| | Max | 1.5 h (90 min) | 4.00 h (240 min) |

### Example 19

### 12.5 mg. Diclofenac K Tablet Comparison

Test Formulations: Diclofenac potassium 12.5 mg film-coated tablets
Reference Formulation: Diclofenac potassium, 12.5 mg immediate release film-coated tablets, Voltaren Dolo® by Novartis Consumer Health
Study design: Single dose, 2-way, crossover randomised on 24 healthy volunteers
Blood samples drawn: 0 (pre-dose), 5, 10, 15, 20, 30, 45, 60, 75, 90 min, 2, 3, 4, 5, 6, 8, 12 h

Assay: LC-MS-MS //LOQ 0.2 ng/ml

| | | Reference, K salt, 12.5 mg, |
|---|---|---|
| | K salt, 12.5 mg, tablets | Voltaren Dolo ® |
| Description | Diclofenac potassium | Diclofenac potassium 12.5 mg |
| | 12.5 mg film-coated | film-coated tablets |
| | tablets | |
| Active | Diclofenac potassium | Diclofenac potassium mg 12.5 |
| ingredient | mg 12.5 | |
| Excipients | Potassium hydrogen | Colloidal anhydrous silica |
| | carbonate mg 5.50 | Lactose |
| | Mannitol mg 76.25 | Maize starch |
| | Sodium laurylsulfate | Sodium starch glycollate |
| | mg 0.25 | Polyvidone |
| | Glycerol dibehenate | Magnesium stearate |
| | mg 1.50 | Microcrystalline cellulose |
| | Crospovidone mg 3.00 | Hydroxypropylmethylcellulose |
| | Magnesium stearate | Titanidioxide (E171) |
| | mg 1.00 | Macrogol |
| | Film Coating Opadry | Polysorbate 80 |
| | Clear (HPMC 2910 and | Maltodextrin |
| | polyethyleneglycol 400) | |
| | mg 2.00 | |
| Total weight | 102.00 mg | |

| | | PK results | |
|---|---|---|---|
| | | Test | |
| | | (K, tablets 12.5. mg) | Reference (K, tablets 12.5 mg) |
| Cₘₐₓ | Mean | 494.09 | 435.80 |
| | SD | 223.36 | 228.92 |
| | CV % | 45.21 | 52.53 |
| | Min | 130.50 | 162.50 |
| | Max | 909.10 | 959.00 |
| AUC | Mean | 331.19 | 330.14 |
| | SD | 71.42 | 84.70 |
| | CV % | 21.56 | 25.66 |
| | Min | 172.54 | 171.38 |
| | Max | 435.39 | 445.72 |
| tₘₐₓ | Mean | 0.35 h (21 min) | 0.48 h (28.8 min) |
| | SD | 0.20 | 0.35 |
| | CV % | 57.14 | 72.92 |
| | Min | 0.17 h (10.2 min) | 0.17 h (10.2 min) |
| | Max | 1.0 h (60 min) | 1.50 h (90 min) |

### Example 20

### 12.5, 25 and 50 mg. Diclofenac K and Diclofenac K Tablet Comparison

Test Formulations:
   T1: Diclofenac sodium 12.5 mg tablets
   T2: Diclofenac sodium 25 mg film-coated tablets
Reference Formulations:
R1: Diclofenac potassium, 25 mg film-coated tablets, Cataflam® by CIBA GEIGY
R2: Diclofenac 50 mg dispersible tablets, Voltarol® by CIBA GEIGY
Study design: Single dose, 4-way, crossover randomised on 24 healthy volunteers
Blood samples drawn: 0 (pre-dose), 5, 10, 15, 20, 30, 45, 60, 90 min, 2, 3, 4, 5, 6, 8, 10 h

Assay: HPLC-UV //LOQ 10 ng/ml

| | | | | Reference, acid as such, |
|---|---|---|---|---|
| | | | Reference, K salt, 50 mg, | 50 mg, Voltarol ® |
| | Na salt, 2 × 12.5 mg, tablets | Na salt, 25 mg, tablets | Cataflam ® tablets | dispersible tablets |
| Description | Diclofenac sodium 12.5 mg | Diclofenac sodium 25 mg | Diclofenac potassium 50 mg | Diclofenac free acid 46.5 mg |
| | film-coated tablets | film-coated tablets | film-coated tablets | dispersible tablets |
| Active | Diclofenac sodium 12.5 mg | Diclofenac sodium 25 mg | Diclofenac potassium 50 mg | Diclofenac acid 46.5 mg |
| ingredient | | | | (equivalent to 50 mg as |
| | | | | sodium salt) |
| Excipients | Potassium bicarbonate mg 5.5 | Potassium bicarbonate | Calcium phosphate | Microcrystalline cellulose |
| | Mannitol mg 25 | mg 11 | Saccharose | Croscarmellose sodium |
| | Lactose mg 23.75 | Mannitol mg 50 | Maize starch | Sodium starch glycollate |
| | Maize starch mg 22.5 | Lactose mg 47.5 | Talc | Sodium saccharin |
| | Cellulose methylether mg | Maize starch mg 45 | Sodium | Cellulose |
| | 0.075 | Cellulose methylether | carboxymethylcellulose | Hydrogenated castor oil |
| | Sodium laurylsulfate mg | mg 0.15 | Colloidal anhydrous | Talc |
| | 0.125 | Sodium laurylsulfate mg | silicium | Silicion dioxide |
| | Polyvinylpyrrolidone mg 3 | 0.25 | Polyvinylpyrrolidone | Erythrosine |
| | Sodium starch glycollate mg 5 | Polyvinylpyrrolidone mg 6 | Microcrystalline cellulose | Aluminium oxide |
| | Magnesium stearate mg 1.5 | Sodium starch glycollate | Magnesium stearate | Blackcurrant flavour |
| | Colloidal hydrated silicium | mg 10 | Polyethylenglycole | |
| | mg 0.55 | Magnesium stearate mg 3 | Titanidioxide (E171) | |
| | Microcrystalline cellulose mg | Colloidal hydrated | Iron oxide red (E172) | |
| | 0.5 | silicium mg 1.1 | | |
| | Film Coating Opadry Red | Microcrystalline | | |
| | (HPMC 2910, | cellulose mg 1 | | |
| | polyethyleneglycol 400) mg | Film Coating Opadry | | |
| | 2.25 | Red (HPMC 2910, | | |
| | | polyethyleneglycol 400) | | |
| | | mg 4.5 | | |
| Total weight | 102.25 | 204.5 mg | | |

| | | PK results | | | |
|---|---|---|---|---|---|
| | | | | | Reference 2 |
| | | | Test 2 (Na, tablets | Reference 1 (K, | (dispersible tablets |
| | | Test 1 (Na, tablets 2 × 12.5 mg) | 25 mg) | tablets 25 mg) | 50 mg)* |
| Cₘₐₓ | Mean | 847.0 | 861.3 | 453.4 | 351.4 |
| | SD | 440.7 | 395.1 | 265.49 | 179.4 |
| | CV % | 0.52 | 0.46 | 0.59 | 0.51 |
| | Min | 314.4 | 221.2 | 1799.9 | 108.9 |
| | Max | 1700.9 | 1569.3 | 992.3 | 635.2 |
| AUC | Mean | 570.50 | 565.59 | 518.46 | 506.26 |
| | SD | 126.23 | 159.77 | 154.54 | 161.92 |
| | CV % | 22 | 26 | 30 | 32 |
| | Min | 346.13 | 304.44 | 322.04 | 322.71 |
| | Max | 859.60 | 911.34 | 836.58 | 858.59 |
| tₘₐₓ | Mean | 0.44 h (26.4 min) | 0.41 h (24.6 min) | 1.19 h (71.4 min) | 0.68 h (40.8 min) |
| | SD | 0.20 | 0.20 | 1.03 | 0.66 |
| | CV % | 46 | 49 | 87 | 96 |
| | Min | 0.17 h (10.2 min) | 0.17 h (10.2 min) | 0.25 h (15 min) | 0.17 h (10.2 min) |
| | Max | 1.0 h (60 min) | 1.00 h (60 min) | 4.00 h (360 min) | 2.00 h (120 min) |

TABLE 1
Pharmacokinetic parameters for two different diclofenac formulations: test (Diclofenac
potassium salt sachets) and reference (Diclofenac potassium salt sugar coated tablets)

| | tₘₐₓ | | Cₘₐₓ | | t_{1/2} | | AUC₀₋₁ | |
|---|---|---|---|---|---|---|---|---|
| | (h) | | (ng/mL) | | (h) | | (ng · mL⁻¹-h) | |
| Vol. No. | Test | Ref. | Test | Ref. | Test | Ref. | Test | Ref. |
| Vol. 1 | 0.250 | 0.500 | 1573.000 | 1186.211 | 1.505 | 0.939 | 1024.511 | 885.549 |
| Vol. 2 | 0.250 | 4.000 | 2382.368 | 965.100 | 0.875 | 1.358 | 1653.124 | 2092.036 |
| Vol. 3 | 0.184 | 1.000 | 2614.655 | 1352.400 | 0.796 | 1.610 | 1687.529 | 1763.484 |
| Vol. 4 | 0.250 | 3.000 | 2404.848 | 735.454 | 0.996 | 1.132 | 1881.944 | 1834.958 |
| Vol. 5 | 0.250 | 0.500 | 2971.457 | 1405.000 | 1.667 | 1.903 | 1819.756 | 1687.075 |
| Vol. 6 | 0.250 | 0.750 | 2158.700 | 1351.500 | 0.843 | 0.650 | 1197.716 | 1091.996 |
| Vol. 7 | 0.250 | 0.750 | 1739.200 | 1741.717 | 0.596 | 0.658 | 1448.713 | 1301.887 |
| Vol. 8 | 0.250 | 0.500 | 1715.350 | 543.300 | 0.818 | 1.111 | 991.864 | 1126.414 |
| Vol. 9 | 0.250 | 0.750 | 444.112 | 747.800 | 0.787 | 1.188 | 669.084 | 886.300 |
| Vol. 10 | 0.267 | 0.750 | 2350.100 | 1110.400 | .0960 | 1.070 | 1327.808 | 1020.286 |
| Vol. 11 | 0.167 | 0.500 | 1867.200 | 1465.502 | 1.141 | 0.762 | 1337.821 | 892.870 |
| Vol. 12 | 0.167 | 0.500 | 4273.026 | 1432.200 | 1.052 | 0.697 | 1703.655 | 1139.003 |
| Vol. 13 | 0.250 | 0.500 | 2097.089 | 1155.371 | 1.313 | 1.198 | 1486.526 | 1233.531 |
| Vol. 14 | 0.167 | 0.250 | 2242.684 | 967.795 | 0.997 | 0.837 | 987.522 | 927.726 |
| Vol. 15 | 0.184 | 0.500 | 2040.247 | 1129.957 | 0.724 | 0.804 | 1213.725 | 1040.424 |
| Vol. 16 | 0.250 | 0.750 | 2143.692 | 818.200 | 0.560 | 1.199 | 1186.603 | 1250.221 |
| Vol. 17 | 0.250 | 1.500 | 1527.845 | 480.900 | 2.752 | 1.309 | 958.821 | 987.797 |
| Vol. 18 | 0.250 | 1.000 | 1859.608 | 666.500 | 1.630 | 1.383 | 1131.413 | 933.008 |
| Vol. 19 | 0.250 | 0.750 | 1537.508 | 770.100 | 1.726 | 1.137 | 980.348 | 906.275 |
| Vol. 20 | 0.250 | 0.250 | 1956.004 | 655.100 | 0.853 | 0.883 | 1309.289 | 1036.836 |
| Vol. 21 | 0.250 | 0.500 | 3551.360 | 2421.060 | 1.322 | 1.233 | 2147.217 | 1639.619 |
| Vol. 22 | 0.167 | 0.500 | 2464.978 | 1274.648 | 0.611 | 0.624 | 1038.817 | 816.924 |
| Vol. 23 | 0.167 | 0.750 | 2304.351 | 453.500 | 2.066 | 0.862 | 1161.414 | 1049.327 |
| Vol. 24 | 0.250 | 0.500 | 2901.504 | 894.337 | 0.970 | 1.279 | 1645.384 | 1086.512 |
| Mean | 0.228 | 0.885 | 2213.370 | 1071.461 | 1.148 | 1.076 | 1332.942 | 1192.544 |
| SD | 0.038 | 0.860 | 743.099 | 450.780 | 0.523 | 0.320 | 358.048 | 350.116 |
| CV % | 16.300 | 97.091 | 33.573 | 42.072 | 45.557 | 29.700 | 26.862 | 29.359 |
| Min. | 0.167 | 0.250 | 444.112 | 453.500 | 0.560 | 0.624 | 669.084 | 816.924 |
| Max. | 0.267 | 4.000 | 4273.026 | 2421.060 | 2.752 | 1.903 | 2147.217 | 2092.036 |
| Geom.Mean | 0.225 | 0.692 | 2070.719 | 987.480 | 1.056 | 1.032 | 1287.195 | 1150.713 |
| Median | 0.250 | 0.625 | 2151.196 | 1039.098 | 0.983 | 1.122 | 1261.507 | 1067.920 |

| | | | | | | | AUC | |
|---|---|---|---|---|---|---|---|---|
| | AUC₀₋₀ | | | | Cₘₐₓ/AUC_{U-o} | | extrapolated | |
| | (ng · mL⁻¹-h) | | C₁ | | (h¹) | | (%) | |
| Vol. No. | Test | Ref. | Test | Ref. | Test | Ref. | Test | Ref. |
| Vol. 1 | 1050.137 | 910.868 | 11.800 | 18.700 | 1.498 | 1.302 | 2.37 | 0.00 |
| Vol. 2 | 1693.172 | 2092.036 | 31.700 | 13.500 | 1.407 | 0.461 | 1.82 | 1.38 |
| Vol. 3 | 1718.755 | 1788.111 | 27.200 | 10.600 | 1.521 | 0.756 | 0.83 | 1.15 |
| Vol. 4 | 1897.754 | 1856.346 | 11.000 | 13.100 | 1.267 | 0.396 | 1.39 | 1.88 |
| Vol. 5 | 1845.486 | 1719.478 | 10.700 | 11.800 | 1.610 | 0.817 | 1.56 | 1.90 |
| Vol. 6 | 1216.693 | 1113.146 | 15.600 | 22.500 | 1.774 | 1.214 | 2.50 | 1.79 |
| Vol. 7 | 1485.867 | 1325.661 | 43.200 | 25.500 | 1.170 | 1.314 | 1.46 | 1.78 |
| Vol. 8 | 1006.522 | 1146.775 | 12.400 | 12.700 | 1.704 | 0.466 | 3.08 | 2.75 |
| Vol. 9 | 690.354 | 911.329 | 18.700 | 14.600 | 0.643 | 0.821 | 1.74 | 1.80 |
| Vol. 10 | 1351.357 | 1038.971 | 17.000 | 12.100 | 1.739 | 1.069 | 3.01 | 3.01 |
| Vol. 11 | 1379.311 | 920.579 | 25.200 | 25.200 | 1.354 | 1.592 | 1.62 | 2.03 |
| Vol. 12 | 1731.709 | 1162.638 | 18.500 | 23.500 | 2.468 | 1.232 | 1.26 | 1.56 |
| Vol. 13 | 1505.454 | 1253.088 | 10.000 | 11.300 | 1.393 | 0.922 | 2.58 | 2.26 |
| Vol. 14 | 1013.665 | 949.163 | 18.200 | 17.700 | 2.212 | 1.020 | 1.91 | 2.86 |
| Vol. 15 | 1237.399 | 1071.029 | 22.700 | 126.400 | 1.649 | 1.055 | 1.33 | 1.58 |
| Vol. 16 | 1202.653 | 1270.280 | 19.900 | 11.600 | 1.782 | 0.644 | 4.16 | 2.80 |
| Vol. 17 | 100.433 | 1006.986 | 10.500 | 14.900 | 1.527 | 0.478 | 5.51 | 2.26 |
| Vol. 18 | 1197.411 | 954.597 | 28.100 | 10.800 | 1.553 | 0.698 | 2.57 | 2.11 |
| Vol. 19 | 1006.229 | 925.835 | 10.400 | 11.900 | 1.528 | 0.832 | 2.03 | 2.02 |
| Vol. 20 | 1336.472 | 1058.242 | 22.400 | 16.800 | 1.464 | 0.619 | 1.19 | 1.07 |
| Vol. 21 | 2173.030 | 1657.372 | 13.500 | 10.000 | 1.634 | 1.461 | 1.75 | 1.68 |
| Vol. 22 | 1057.293 | 830.908 | 21.000 | 15.500 | 2.331 | 1.534 | 3.13 | 1.80 |
| Vol. 23 | 1198.950 | 1068.588 | 12.600 | 15.500 | 1.922 | 0.424 | 2.19 | 1.94 |
| Vol. 24 | 1682.290 | 1108.024 | 26.400 | 11.700 | 1.725 | 0.807 | 2.10 | 1.78 |
| Mean | 1361.600 | 1214.169 | 19.113 | 15.725 | 1.620 | 0.914 | 2.213 | 1.883 |
| SD | 358.359 | 348.108 | 8.244 | 5.160 | 0.377 | 0.365 | 1.035 | 0.641 |
| CV % | 26.319 | 28.671 | 43.134 | 32.812 | 23.277 | 39.991 | 46.795 | 34.056 |
| Min. | 690.354 | 830.908 | 10.000 | 10.000 | 0.643 | 0.396 | 0.833 | 0.000 |
| Max. | 2173.030 | 2092.036 | 43.200 | 26.400 | 2.468 | 1.592 | 5.512 | 3.010 |
| Geom.Mean | 1316.580 | 1173.325 | 17.609 | 15.011 | 1.573 | 0.841 | 2.023 | // |
| Median | 1286.936 | 1089.527 | 18.350 | 14.050 | 1.582 | 0.827 | 1.974 | 1.843 |

1) A method of treating acute pain comprising administering an immediate release oral dosage form comprising diclofenac potassium together with one or more alkali metal bicarbonates and customary excipients and adjuvants, wherein said alkali metal bicarbonates are present in an amount of greater than about 20% by weight based on the weight of diclofenac.

2) The method of claim 1 wherein said one or more alkali metal carbonates or bicarbonates are present in an amount of from about 20 to about 80% by weight based on the weight of diclofenac.

3) The method of claim 1 wherein said dosage form is a tablet comprising about 50 mg. of diclofenac potassium.

4) The method of claim 1 wherein said dosage form is a capsule comprising about 50 mg. of diclofenac potassium.

5) The method of claim 1 wherein said dosage form is a powder dosage form comprising about 50 mg. of diclofenac potassium dissolved or dispersed in water.

6) The method of claim 1 wherein said dosage form is a tablet comprising 25 mg. of diclofenac potassium.

7) The method of claim 1 wherein said dosage form is a capsule comprising 25 mg. of diclofenac potassium.

8) The method of claim 1 wherein said dosage form is a powder dosage form comprising 25 mg. of diclofenac potassium dissolved or dispersed in water.

9) The method of claim 1 wherein said dosage form is a tablet comprising 12.5 mg. of diclofenac potassium.

10) The method of claim 1 wherein said dosage form is a capsule comprising 12.5 mg. of diclofenac potassium.

11) The method of claim 1 wherein said dosage form is a powder dosage form comprising 12.5 mg. of diclofenac potassium dissolved or dispersed in water.

12) A method of treating acute pain comprising orally administering an immediate release oral dosage form comprising diclofenac in acid and/or salt form together with one or more alkali metal carbonates or bicarbonates and customary excipients and adjuvants, wherein said one or more alkali metal carbonates or bicarbonates are present in an amount of greater than about 20% by weight based on the weight of diclofenac.

13) The method of claim 12 wherein said one or more alkali metal carbonates or bicarbonates are present in an amount of from about 20 to about 80% by weight based on the weight of diclofenac.

14) The method of claim 12 wherein said dosage form is a tablet.

15) The method of claim 12 wherein said dosage form is a capsule.

16) The method of claim 12 wherein said dosage form is a powder dosage form dissolved or dispersed in water.

17) A method of treating acute pain comprising administering an immediate release pharmaceutical dosage form for oral use comprising diclofenac in acid and/or salt form and means for decreasing, average Tₘₐₓ and increasing average Cₘₐₓ of said diclofenac.

18) The method of claim 17 wherein said dosage form is a tablet comprising about 50 mg. of diclofenac potassium.

19) The method of claim 17 wherein said dosage form is a capsule comprising about 50 mg. of diclofenac potassium.

20) The method of claim 17 wherein said dosage form is a powder dosage form comprising about 50 mg. of diclofenac potassium dissolved or dispersed in water.

21) The method of claim 17 wherein said dosage form is a tablet comprising 25 mg. of diclofenac potassium.

22) The method of claim 17 wherein said dosage form is a capsule comprising 25 mg. of diclofenac potassium.

23) The method of claim 17 wherein said dosage form is a powder dosage form comprising 25 mg. of diclofenac potassium dissolved or dispersed in water.

24) The method of claim 17 wherein said dosage form is a tablet comprising 12.5 mg. of diclofenac potassium.

25) The method of claim 17 wherein said dosage form is a capsule comprising 12.5 mg. of diclofenac potassium.

26) The method of claim 17 wherein said dosage form is a powder dosage form comprising 12.5 mg. of diclofenac potassium dissolved or dispersed in water.

27) The method of claim 17 wherein said dosage form is a tablet comprising about 50 mg. of diclofenac sodium.

28) The method of claim 17 wherein said dosage form is a capsule comprising about 50 mg. of diclofenac sodium.

29) The method of claim 17 wherein said dosage form is a powder dosage form comprising about 50 mg. of diclofenac sodium dissolved or dispersed in water.

30) The method of claim 17 wherein said dosage form is a tablet comprising 25 mg. of diclofenac sodium.

31) The method of claim 17 wherein said dosage form is a capsule comprising 25 mg. of diclofenac sodium.

32) The method of claim 17 wherein said dosage form is a powder dosage form comprising 25 mg. of diclofenac sodium dissolved or dispersed in water.

33) The method of claim 17 wherein said dosage form is a tablet comprising 12.5 mg. of diclofenac sodium.

34) The method of claim 17 wherein said dosage form is a capsule comprising 12.5 mg. of diclofenac sodium.

35) The method of claim 17 wherein said dosage form is a powder dosage form comprising 12.5 mg. of diclofenac sodium dissolved or dispersed in water.

36) The method of claim 17 wherein said dosage form is a tablet comprising about 50 mg. of diclofenac potassium, and said tablet exhibits an average Cₘₐₓ of from about 1750 to about 2000 and an average Tₘₐₓ of from about 12 to about 25 minutes.

37) The method of claim 17 wherein said dosage form is a powder sachet comprising about 50 mg. of diclofenac potassium, and said sachet exhibits an average Cₘₐₓ of from about 1500 to about 2000 and an average Tₘₐₓ of from about 8 to about 20 minutes.

38) The method of claim 17 wherein said dosage form is a powder sachet comprising about 50 mg. of diclofenac potassium, and said sachet exhibits an average Cₘₐₓ of from about 1900 to about 2500 and an average Tₘₐₓ of from about 8 to about 20 minutes.
